(19) United States
(12) Patent Application Publication
(10) Pub. No.: US 2026/0274809 A1
(43) Pub. Date: Sep. 17, 2026

(54) TWO-STAGE CATALYTIC AMINATION-CYCLIZATION PROCESS FOR THE PRODUCTION OF PIPERAZINE AND SUBSTITUTED PIPERAZINE

(71) Applicant: UOP LLC, Des Plaines, IL (US)

(72) Inventors: Nabil Al Yassir, Ontario (CA); Carl J. Stevens, Seaside, CA (US); Edwin P. Boldingh, Arlington Heights, IL (US)

(21) Appl. No.: 19/437,671

(22) Filed: Dec. 31, 2025

Related U.S. Application Data
(60) Provisional application No. 63/771,948, filed on Mar. 14, 2025.

(51) Int. Cl. C07D 295/00

(52) U.S. Cl. CPC C07D 295/00

(57) ABSTRACT

Processes for the production of piperazine and alkylated piperazine (2-MethylPiperazine) are described. The processes involve forming selective aminated intermediates in situ using a two-stage reactor system. The amination reaction zone is designed to form a fully or partially aminated product, while the cyclization reactor is tailored for the inter/intramolecular cyclization of the previously formed aminated intermediate.

### RELATED APPLICATIONS

This application claims priority to U.S. Provisional Patent Application Ser. No. 63/771,948, filed on Mar. 14, 2025, the entirety of which is incorporated herein by reference.

### BACKGROUND

Cyclic diamine piperazine is one of the leading solvent candidate for CO₂ capture processes from post-combustion sources, such as flue gas, offered by multiple technology providers. Many other solvents are not suitable because they have lower CO₂ capacity and high levels of thermal and oxidative degradation that prevent operation at high temperatures.

Currently, piperazine is only made as a by-product of ethylene amines commercial technologies (viz. reductive amination of ethylene dichloride (EDC) or monoethanolamine (MEA)). These processes have limited yield (e.g., a maximum of 10% of the total products) and limited market supply participants.

US Application No. 2014/0371452 describes a method for the reductive amination of diethanolamine to form a product composition that includes piperazine and aminoethylethanolamine along with other by-products. A catalyst with a transitional alumina/second metal oxide support and a mixture of catalytic metal is used for the reaction. As shown in Examples 1-3 and Tables 2-4, the combined amount of piperazine and aminoethylethanolamine in the product composition is 75 wt % or greater of the products. The piperazine in the product composition is 10-40 wt % of the products.

The market growth of ethylene amines is about 3.7% compound annual growth rate (CAGR) whereas piperazine growth is about 10-20% CAGR. It is expected that piperazine capacity will be insufficient to meet the demand if the market adopts piperazine for carbon capture processes.

Therefore, there is a need for processes to produce piperazine having increased yield and decreased byproducts.

### BRIEF DESCRIPTION OF THE DRAWINGS

FIG. 1 illustrates one embodiment of the process of the present invention.

### DESCRIPTION

U.S. Application Ser. No. 63/737,268, entitled Catalytic Method for Selective Cyclo-Amination of Alkanolamine and Diol to Produce On-Purpose Cyclic Ethyleneamine of Piperazine and Derivative, filed Dec. 12, 2024, describes a catalytic process for making piperazine and substituted piperazine. The process involves the reaction of an alkanolamine or an ethyleneamine or a diol in a reaction zone comprising a reactor in the presence of anhydrous ammonia and a catalyst comprising a 10-member ring zeolite to form a reaction mixture comprising piperazine. U.S. Application Ser. No. 63/737,390, entitled Process for the Production of a Co-Mixture of Piperazine and Methyl-Substituted Piperazine, filed Dec. 20, 2024, describes a process for making a co-mixture of piperazine and methyl-substituted piperazine. The process involves co-feeding a linear alkanolamine, or a diol, or an ethyleneamine with an analog of the alkanolamine, or the diol, or the ethyleneamine in a reaction zone comprising a reactor in the presence of anhydrous ammonia and a catalyst comprising a 10-member ring zeolite to produce a reaction mixture comprising the piperazine and the methyl-substituted piperazine.

While these processes successfully produce piperazine and mixtures of piperazine and methyl-substituted piperazine, respectively, with increased selectivity compared with the prior art processes, further improvements in selectivity are desirable.

The present invention addresses the challenges of obtaining piperazine selectivity greater than 50%.

In a single stage reactor, multiple reactions take place simultaneously, including amination, cyclization, secondary condensation, elimination, and the like. As a result, it is difficult to control the formation of desirable cyclization intermediates. Even when the proper intermediate is formed, given the high severity of the reaction conditions, it could undergo a side-reaction and not form piperazine.

Although starting with a fully aminated feed, such as aminoethylethanolamine (AEEA), ethylenediamine (EDA), or propylenediamine (PDA) (PDA can be used as the C3 analog for a piperazine alkylated additive) would be ideal, the high cost of the raw materials would make the process uneconomical.

The present process is designed to overcome these limitations. It does so by forming the highly selective aminated intermediates in situ using a two-stage reactor system. The first reactor, the amination reactor, is designed to form a partially or fully aminated product, while the second reactor, the cyclization reactor, is tailored for the inter/intramolecular cyclization of the previously formed aminated intermediate.

The advantages of the present process include the production of a highly efficient intermediate for piperazine and 2-methylpiperazine (the piperazine additive) which avoids the need for an expensive feed for cyclization reactor or the C3 analog. In addition, the low severity operation for the cyclization reactor avoids the formation of undesirable side-products, resulting in a highly selective process for making piperazine.

The presence of the amination reactor enables the formation of desirable intermediates, such as AEEA in the case of diethanolamine (DEA) feed, or ethylenediamine (EDA) in the case of monoethanolamine (MEA) or ethylene glycol (EG) feed. Propylenediamine (a C3 analog) can be made from monopropylene glycol (MPG), which is an inexpensive (commodity) feed.

The aminated intermediates are cyclized in the cyclization reaction zone.

One aspect of the invention is a process for the production of piperazine. In one embodiment, the process comprises aminating a feed stream comprising an alkanolamine, or a diol, or both in an amination reaction zone comprising an amination reactor in the presence of anhydrous ammonia and an amination catalyst to provide an aminated feed stream comprising aminated alkanolamine, or an aminated diol, or both; and cyclizing the aminated feed stream in a cyclization reaction zone comprising a cyclization reactor in the presence of a cyclization catalyst comprising a 10-member ring zeolite to produce a reaction mixture comprising piperazine.

In some embodiments, the amination catalyst comprises a 10-member ring MFI zeolite, or a 12-member ring FAU zeolite, or amorphous silica-alumina, or combinations thereof. In some embodiments, the 12-member ring FAU zeolite or the amorphous silica-alumina is modified with phosphorus. In some embodiments, the phosphorus comprises phosphoric acid, phosphorous oxide, ammonium dihydrogen phosphate, phosphomolybdic acid, or combinations thereof. In some embodiments, the 12-member ring FAU zeolite is dealuminated. In some embodiments, the 10-member ring MFI zeolite is modified by steaming.

In some embodiments, the amination catalyst has an acid sites in a range of 0.40 to 1.15 mmol/g. The catalyst has a high ratio of weak acid sites to strong acid sites, which primarily promotes the amination reaction without cyclization.

In some embodiments, the amination catalyst has a mole ratio of Si to Al in a range of 5-140.

In some embodiments, the amination reaction takes place at a temperature in a range of 280° C. to 350° C., or a pressure in a range of 1700-2500 psig, or both.

The feed to the amination reaction zone comprises an alkanolamine, or a diol, or combinations thereof. The characteristics needed for the alkanolamine, or diol are feed reactivity and the level of NH₂ containing groups. Raw material cost is also a factor in the selection.

Any suitable alkanolamine can be used. In some embodiments, the alkanolamine comprises diethanolamine, or monoethanolamine, or triethanolamine, or monoisopropanolamine, or aminoethylethanolamine, or combinations thereof.

Any suitable diol can be used. In some embodiments, the diol comprises ethylene glycol or monopropylene glycol, or combinations thereof.

In some embodiments, the feed stream further comprises a C3 analog of the alkanolamine or the diol, wherein the aminated feed stream further comprises an aminated C3 analog, and wherein the reaction mixture further comprises an alkylated piperazine.

In some embodiments, the C3 analog comprises monopropylene glycol, or monoisopropanol amine, or propylenediamine, or combinations thereof.

An anhydrous ammonia stream is injected into the amination reaction zone to convert the hydroxyl groups in the feed to NH₂ groups.

The aminated feed stream from the amination reaction zone is sent to the cyclization reaction zone where the aminated intermediates are cyclized to form piperazine and optionally, alkylated piperazine, such as methylpiperazine.

In some embodiments, the cyclization catalyst comprises a 10-member ring zeolite. In some embodiments, the 10-member ring zeolite comprises an MFI-type zeolite. Suitable 10-member ring zeolites include, but are not limited to, ZSM-5, MEL zeolites, MTT zeolites, TUN zeolites, and MWW zeolites.

In some embodiments, the 10-member ring zeolite is modified with phosphorus. The phosphorus may comprise phosphoric acid, phosphorous oxide, ammonium dihydrogen phosphate, phosphomolybdic acid, or combinations thereof.

In some embodiments, the 10-member ring zeolite is modified by silylation. The external surface of the zeolite is passivated through the chemical liquid deposition of silica. The silica deposition can be repeated one or more times, typically from 1 to 6 depositions. The zeolite form can be in H form or ammonium (NH₄) form.

In some embodiments, the 10-member ring zeolite is modified with gallium. The 10-member ring zeolite may comprise gallium in the range of 0.5 wt % to 5 wt %.

In some embodiments, the catalyst has a mole ratio of Si to Al in a range of 10-140.

In some embodiments, the catalyst has an acidity in a range of 0.75 to 1.20 mmol/g.

In some embodiments, the alkanolamine comprises diethanolamine and the mole ratio of anhydrous ammonia to diethanolamine is in a range of 10 to 100.

In some embodiments, the cyclization reaction takes place at a temperature in a range of 300° C. to 350° C., or a pressure in a range of 1700-2500 psig, or both.

In some embodiments, the reaction mixture from the cyclization reaction zone further comprises a piperazine derivative, and the process further comprises: separating the reaction mixture into a piperazine stream comprising the piperazine, and mono-alkylated piperazine, and a byproduct stream comprising piperazine derivatives and byproducts; and recycling the byproduct stream to the cyclization reaction zone. In some embodiments, the mono-alkylated piperazine comprises 2-methyl piperazine. In some embodiments, the piperazine derivatives comprise heavy alkylated piperazine (more than one alkyl group), or triethylenediamine, or aminoethylpiperazine, or hydroxyethylpiperazine, or combinations thereof. Byproducts comprise heavy ethylene amines, including, but not limited to, diethylene triamine, diethyleneamine, diethylene tetraamine, or combinations thereof.

Another aspect of the invention is a process for the production of piperazine. In one embodiment, the process comprises aminating a feed stream comprising an alkanolamine, or a diol, or both, and optionally a C3 analog of the alkanolamine or the diol or both in an amination reaction zone comprising an amination reactor in the presence of anhydrous ammonia and an amination catalyst to provide an aminated feed stream comprising an aminated alkanolamine, or an aminated diol, or both and optionally an aminated C3 analog of the alkanolamine or the diol or both, wherein the amination catalyst comprises a 10-member ring MFI zeolite, a 12-member ring FAU zeolite, or amorphous silica-alumina; cyclizing the aminated feed stream in a cyclization reaction zone comprising a cyclization reactor in the presence of a cyclization catalyst comprising a 10-member ring zeolite to produce a reaction mixture comprising piperazine, and optionally an alkylated piperazine, and optionally a piperazine derivative; and separating the reaction mixture into a piperazine stream comprising the piperazine and optionally the alkylated piperazine, and a byproduct stream comprising the piperazine derivative.

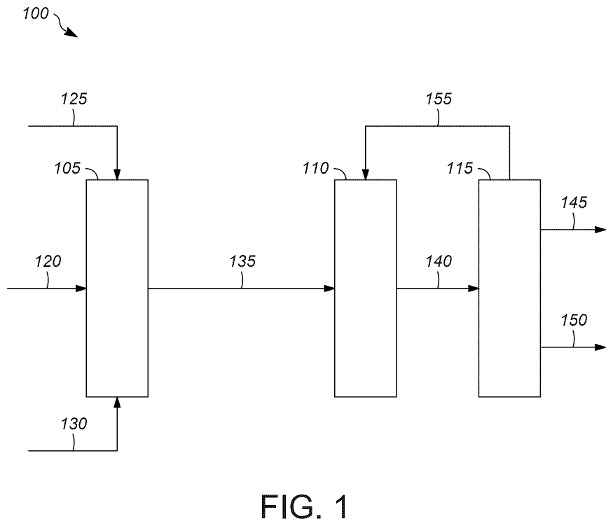

FIG. 1 illustrates one embodiment of the process 100 for producing piperazine. The process 100 includes an amination reaction zone 105, a cyclization reaction zone 110, and a separation zone 115.

The feed stream 120 is sent to the amination reaction zone 105. The feed stream 120 comprises an alkanolamine, or a diol, such as ethylene glycol, or monoethanolamine, or diethanolamine, for example.

The amination reaction zone 105 contains an amination catalyst comprising a 10-member ring MFI zeolite, a 12-member ring FAU zeolite, or amorphous silica-alumina, as described above.

An anhydrous ammonia stream 125 is injected into the amination reaction zone 105 to convert the hydroxyl groups in the feed to NH₂ groups.

A C3 analog 130 (e.g., (e.g. momopropylene glycol (MPG), or monoisopropanol amine (MIPA)) can also be introduced into the amination reaction zone 105 if an alkylated piperazine is to be prepared.

The amination reaction produces an aminated intermediate stream 135. For example, if the feed is monoethanolamine or ethylene glycol, the aminated intermediate stream 135 will comprise EDA, while if the feed stream comprises diethanolamine, AEEA will be produced. If the C3 analog feed stream comprises MPG, EDA, MIPA, and/or propylenediamine (PDA) will be produced, while if C3 analog feed stream comprises MIPA, EDA and PDA will be produced.

The aminated intermediate stream 135 is sent to the cyclization reaction zone 110 where the aminated intermediates are converted to piperazine or alkylated piperazine.

The cyclized effluent stream 140 comprising piperazine and/or alkylated piperazine, and/or piperazine derivatives and/or heavy ethylene amines, and/or unconverted feed. The cyclized effluent stream 140 is sent to the separation zone 115 where it is separated into a piperazine stream 145 comprising piperazine, and alkylated piperazine stream 150 comprising alkylated (2-MethylPiperazine) piperazine, and a recycle stream 155 comprising piperazine derivatives, and any unconverted feed. Other separation schemes could be used. For example, the piperazine and mono-alkylated piperazine could be in one stream, while the recycle stream could contain the heavy alkylated piperazine, piperazine derivatives, and any unconverted feed.

### EXAMPLES

The following examples illustrate aspects of the invention. Example 1 and 2 describe two-stage process to obtain primarily aminated feed through amination reactor followed by cyclic piperazine and alkylated piperazine via cyclization reactor. The comparative example shows simultaneous synthesis of piperazine and alkylated piperazine.

### Example 1

Selective amination of ethylene glycol and monopropylene glycol (MPG) was conducted in a high pressure stirred SS316 batch reactor. The catalyst, which is added in powdered form, was activated ex. situ at 275° C. for 10 hours under nitrogen flow. Examples of amination catalyst include amorphous silica alumina (ASA, SiO2/Al2O3=2), Phosphorous (P) modified ASA, phosphomolybdic acid (PMA) modified ASA, and FAU zeolite (USY). Examples of cyclization catalyst include zeolite of variable SiO₂/Al₂O₃ ratio (23-280) (MFI23, 23 represents SiO₂/Al₂O₃); large (2 microns) MFI crystals; FAU zeolite (HY5, USY30), metal-containing MFI (Gallium (Ga)), steamed MFI zeolite, and silylated (SiO2 passivated) MFI. A certain amount of feed and liquified anhydrous ammonia were charged into the vessel, with an NH₃ to (feed) of 10-100 (molar ratio). The mixture was heated (280-350° C.) under stirring for 30 min to 10 hours on stream under autogenic pressure (1700-2500 psig). Liquid products were analyzed offline using gas chromatography equipped with flame ionization detector (FID) and CP-Volamine 60 m×0.32 mm i.d×5 μm.

Aminated products, such as monoethanolamine (MEA) and fully aminated ethylenediamine (EDA), are the only products from the amination of diol (e.g., cost-effective ethylene glycol) using amination catalyst of weak Bronsted acid sites (Table 1). Cyclic products (e.g., piperazine (PZ) and PZ derivatives) are not formed. Upon amination of C3 analogs (e.g., monopropylene glycol (MPG)), products with different level of amination are formed, i.e., partially aminated monoisopropanolamine (MIPA), and fully aminated products such as ethylenediamine (EDA) and propylenediamine (PDA). This is dependent on the amination catalyst and process parameters (e.g. molar ratio of anhydrous NH3 to feed). This shows that it is possible to limit the reaction to only aminated products.

TABLE 1
Amination of ethylene glycol (EG) and C3 analog monopropylene glycol
(MPG) (3 HOS, 150 barg, 300° C.) in the amination reactor

| | C3 | Amination | NH3/feed | χ | Selectivity (%) | | | |
|---|---|---|---|---|---|---|---|---|
| Feed | analog | Catalyst | (molar) | (%)^{a} | MEA | EDA | MIPA | PDA |
| EG | — | ASA | 50 | 6.80 | 66.5 | 33.5 | — | — |
| | — | P₂O₅/ASA | 50 | 31.1 | 34.2 | 65.8 | — | — |
| | | (11.5% | | | | | | |
| | | P₂O₅) | | | | | | |
| | — | PMA/ASA | 50 | 15.6 | 44.9 | 55.1 | — | — |
| | | (5% MoO₃, | | | | | | |
| | | 0.2% P₂O₅) | | | | | | |
| | — | P₂O₅/USY | 50 | 18.9 | 21.7 | 78.3 | — | — |
| | | (11.5% | | | | | | |
| | | P₂O₅) | | | | | | |
| — | MPG | ASA | 65 | 27.5 | — | 68.5 | 31.5 | — |
| | MPG | P₂O₅/ASA | 65 | 17.6 | — | 45.5 | — | 54.5 |
| | | (11.5% | | | | | | |
| | | P₂O₅) | | | | | | |

^{a}Conversion

### Example 2

Cyclization of fully aminated ethylenediamine (EDA) and C3 analog propylenediamine (PDA) which are formed via the animation reactor (Table 1) was carried out similar to the Example 1 procedures. The product composition of EDA cyclization shows predominantly cyclic products of the desirable piperazine and piperazine derivatives. Piperazine selectivity is about 50.7%-64.1%, depending on the catalyst and post-modification. Ga containing MFI40 showed the highest selectivity to PZ. Silylation of MFI zeolite increased PZ selectivity to 60.5% (up from 55.7%), depending on the Si/Al ratio and other factors. Higher yield of piperazine is obtained for the large MFI30 crystals silylated in the NH₄ form, compared to the same process using H form. Similar trends were noted for MFI zeolites with Si/Al ratios ranging from 40 to 140. Co-mixture of piperazine and 2-methylpiperazine (PZ+2-MPZ) is obtained upon the cyclization of fully aminated feed (EDA) and C3 analog (PDA). The selectivity to (PZ+2-MPZ) is about 79.5%, which increased (15% increase) upon co-feeding anhydrous NH₃ (NH₃/EDA (molar)=55) to the cyclization reactor.

TABLE 2
Cyclization of ethylenediamine (EDA) and mixture containing ethylenediamine
(EDA) and propylenediamine (PDA) formed from the amination of ethylene
glycol and monopropylene glycol (3 HOS, 150 barg, 300° C.)

| Aminated Feed | | | | Selectivity (%) | | | | | |
|---|---|---|---|---|---|---|---|---|---|
| | C3 | Cyclization | χ | | 2- | PZ + 2- | | | |
| Feed | analog | Catalyst | (%)^{a} | PZ | MPZ | MPZ | TEDA | AEP | DETA |
| EDA | — | 2% Ga/MFI40 | 39.3 | 64.1 | — | — | 12.4 | 23.5 | — |
| | — | 2% Ga/MFI40 | 71.6 | 57.1 | — | — | 10.2 | 28.8 | 3.93 |
| | | (6)^{b} | | | | | | | |
| | — | MFI23 ^{c} | 54.2 | 51.9 | — | — | 23.1 | 21.8 | 3.20 |
| | — | MFI23 (n = 1, | 2.10 | — | — | — | 100 | — | — |
| | | 6% SiO2) | | | | | | | |
| | — | MFI30L^{d} | 54.1 | 55.7 | — | — | 14.8 | 20.8 | 8.70 |
| | — | MFI30L^{d} (n = | 18.7 | 55.1 | — | — | 30.2 | — | 14.7 |
| | | 1, 6% SiO2) | | | | | | | |
| | — | NH4MFI30L^{d} | 56.6 | 60.5 | — | — | 6.90 | 10.2 | 22.4 |
| | | (n = 1, 6% | | | | | | | |
| | | SiO₂) | | | | | | | |
| | — | MFI80 | 66.1 | 50.7 | — | — | 28.2 | 18.3 | 2.76 |
| | — | NH4MFI80 (n = | 68.0 | 54.6 | — | — | 20.5 | 22.2 | 2.68 |
| | | 1, 6% SiO₂) | | | | | | | |
| EDA | PDA^{e} | MFI30L^{d} | 19.7 | 29.2 | 50.3 | 79.5 | 10.6 | 9.90 | — |
| EDA | PDA^{e} | MFI30L^{d} | 60.4 | 26.7 | 64.9 | 91.6 | 3.20 | 5.20 | — |
| | | (55)^{b} | | | | | | | |

^{a}Conversion;
^{b}number in parentheses represents NH3/EDA molar ratio;
^{c} 1 HOS;
^{d}large crystals;
^{e}wt. % ratio of feed/C3 analog (50/50)

### Comparative Example 1

One-step amination and cyclization of ethylene glycol and C3 analog monopropylene glycol was carried out similar to Example 1 procedures. Piperazine selectivity over non-aminated ethylene glycol is 38.7%, compared to 55.7% for the fully aminated feed ethylenediamine using large MFI30 crystals (MFI30L). Selectivity to (PZ+2-MPZ) over non-aminated mixture of (ethylene glycol+monopropylene glycol) (50/50) is 35% lower than starting with equivalent fully aminated mixture (ethylenediamine+propylenediamine), using MFI30L and comparable molar ratio of anhydrous NH₃/feed.

TABLE 3
Simultaneous one-step amination and cyclization of mixture
containing diol and C3 analog (3 HOS, 150 barg, 300° C.)

| | | | | Selectivity (%) | | | | | | | |
|---|---|---|---|---|---|---|---|---|---|---|---|
| | C3 | Cyclization | χ | | 2- | PZ + 2- | | | | | |
| Feed | analog | Catalyst | (%)^{a} | PZ | MPZ | MPZ | TEDA | AEP | EDA | DETA | MEA |
| EG | — | MFI30L^{b} | 16.0 | 38.7 | — | — | 8.60 | 7.03 | 35.7 | — | 9.97 |
| | | (Pristine) | | | | | | | | | |
| EG | MPG ^{c, d} | MFI30L^{b} | 7.40 | 29.1 | 29.1 | 58.2 | — | — | 41.8 | — | — |
| | | (Pristine) | | | | | | | | | |

^{a}Conversion;
^{b}large crystals;
^{c} wt. % ratio of feed/C3 analog (50/50);
^{d} NH₃/EG (45)

### SPECIFIC EMBODIMENTS

While the following is described in conjunction with specific embodiments, it will be understood that this description is intended to illustrate and not limit the scope of the preceding description and the appended claims.

A first embodiment of the invention is a process for the production of piperazine comprising aminating a feed stream comprising an alkanolamine, or a diol, or both, in an amination reaction zone comprising an amination reactor in the presence of anhydrous ammonia and an amination catalyst to provide an aminated feed stream comprising aminated alkanolamine, or an aminated diol, or both; and cyclizing the aminated feed stream in a cyclization reaction zone comprising a cyclization reactor in the presence of a cyclization catalyst comprising a 10-member ring zeolite to produce a reaction mixture comprising piperazine. An embodiment of the invention is one, any or all of prior embodiments in this paragraph up through the first embodiment in this paragraph wherein the amination catalyst comprises 10-member ring MFI zeolite, or a 12-member ring FAU zeolite, or amorphous silica-alumina, or combinations thereof. An embodiment of the invention is one, any or all of prior embodiments in this paragraph up through the first embodiment in this paragraph wherein the 12-member ring FAU zeolite or the amorphous silica-alumina is modified with phosphorus. An embodiment of the invention is one, any or all of prior embodiments in this paragraph up through the first embodiment in this paragraph wherein the phosphorus comprises phosphoric acid, phosphorous oxide, ammonium dihydrogen phosphate, phosphomolybdic acid, or combinations thereof. An embodiment of the invention is one, any or all of prior embodiments in this paragraph up through the first embodiment in this paragraph wherein the 12-member ring FAU zeolite is dealuminated. An embodiment of the invention is one, any or all of prior embodiments in this paragraph up through the first embodiment in this paragraph wherein the 10-member ring MFI zeolite is modified by steaming. An embodiment of the invention is one, any or all of prior embodiments in this paragraph up through the first embodiment in this paragraph wherein the amination catalyst has acid sites in a range of 0.40 to 1.15 mmol/g. An embodiment of the invention is one, any or all of prior embodiments in this paragraph up through the first embodiment in this paragraph wherein the amination catalyst has a mole ratio of Si to Al in a range of 5-140. An embodiment of the invention is one, any or all of prior embodiments in this paragraph up through the first embodiment in this paragraph wherein the amination reaction takes place at a temperature in a range of 280° C. to 350° C., or a pressure in a range of 1700-2500 psig, or both. An embodiment of the invention is one, any or all of prior embodiments in this paragraph up through the first embodiment in this paragraph wherein the cyclization catalyst comprises a 10-member ring zeolite. An embodiment of the invention is one, any or all of prior embodiments in this paragraph up through the first embodiment in this paragraph wherein the 10-membered ring zeolite comprises a 10-member ring MFI zeolite. An embodiment of the invention is one, any or all of prior embodiments in this paragraph up through the first embodiment in this paragraph wherein the 10-member ring zeolite is modified with gallium, or by silylation, or by steaming, or combinations thereof. An embodiment of the invention is one, any or all of prior embodiments in this paragraph up through the first embodiment in this paragraph wherein the alkanolamine comprises diethanolamine, or monoethanolamine, or triethanolamine, or monoisopropanolamine, or aminoethylethanolamine, combinations thereof. An embodiment of the invention is one, any or all of prior embodiments in this paragraph up through the first embodiment in this paragraph wherein the diol comprises ethylene glycol or monopropylene glycol or both. An embodiment of the invention is one, any or all of prior embodiments in this paragraph up through the first embodiment in this paragraph wherein the feed stream further comprises a C3 analog of the alkanolamine or the diol or both, wherein the aminated feed stream further comprises an aminated C3 analog, and wherein the reaction mixture further comprises an alkylated piperazine. An embodiment of the invention is one, any or all of prior embodiments in this paragraph up through the first embodiment in this paragraph wherein the C3 analog comprises monopropylene glycol or monoisopropanol amine or both. An embodiment of the invention is one, any or all of prior embodiments in this paragraph up through the first embodiment in this paragraph wherein the reaction mixture further comprises a piperazine derivative and further comprising separating the reaction mixture into a piperazine stream comprising the piperazine, and the alkylated piperazine, and a byproduct stream comprising the piperazine derivative; and recycling the byproduct stream to the cyclization reaction zone. An embodiment of the invention is one, any or all of prior embodiments in this paragraph up through the first embodiment in this paragraph wherein the piperazine derivative comprises at least one of a heavy alkylated piperazine, triethylenediamine, aminoethylpiperazine, and hydroxyethylpiperazine. An embodiment of the invention is one, any or all of prior embodiments in this paragraph up through the first embodiment in this paragraph further comprising: co-feeding anhydrous NH₃ to the cyclization reaction zone; and wherein the cyclization reaction takes place at a temperature in a range of 280° C. to 350° C., a pressure in a range of 1700-2500 psig, and a molar ratio of NH₃ to feed of 10 to 100.

A second embodiment of the invention is a process for the production of piperazine comprising aminating a feed stream comprising an alkanolamine, or a diol, or both, and optionally a C3 analog of the alkanolamine or the diol in an amination reaction zone comprising an amination reactor in the presence of anhydrous ammonia and an amination catalyst to provide an aminated feed stream comprising an aminated alkanolamine, or an aminated diol, or both, and optionally an aminated C3 analog of the alkanolamine or the diol or both, wherein the amination catalyst comprises a 10-member ring MFI zeolite, a 12-member ring FAU zeolite, or amorphous silica-alumina; cyclizing the aminated feed stream in a cyclization reaction zone comprising a cyclization reactor in the presence of a cyclization catalyst comprising a 10-member ring zeolite to produce a reaction mixture comprising piperazine, and optionally an alkylated piperazine, and optionally a piperazine derivative; and separating the reaction mixture into a piperazine stream comprising the piperazine and optionally the alkylated piperazine, and a byproduct stream comprising the piperazine derivative. An embodiment of the invention is one, any or all of prior embodiments in this paragraph up through the second embodiment in this paragraph An embodiment of the invention is one, any or all of prior embodiments in this paragraph up through the second embodiment in this paragraph wherein the amination catalyst comprises 10-member ring MFI zeolite, a 12-member ring FAU zeolite, or amorphous silica-alumina; or wherein the cyclization catalyst comprises a 10-member ring zeolite, optionally is modified with gallium, or by silylation, or by steaming; or both

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for the production of piperazine comprising:
aminating a feed stream comprising an alkanolamine, or a diol, or both, in an amination reaction zone comprising an amination reactor in the presence of anhydrous ammonia and an amination catalyst to provide an aminated feed stream comprising aminated alkanolamine, or an aminated diol, or both; and
cyclizing the aminated feed stream in a cyclization reaction zone comprising a cyclization reactor in the presence of a cyclization catalyst comprising a 10-member ring zeolite to produce a reaction mixture comprising piperazine.

2. The process of claim 1 wherein the amination catalyst comprises 10-member ring MFI zeolite, or a 12-member ring FAU zeolite, or amorphous silica-alumina, or combinations thereof.

3. The process of claim 2 wherein the 12-member ring FAU zeolite or the amorphous silica-alumina is modified with phosphorus.

4. The process of claim 3 wherein the phosphorus comprises phosphoric acid, phosphorous oxide, ammonium dihydrogen phosphate, phosphomolybdic acid, or combinations thereof.

5. The process of claim 3 wherein the 12-member ring FAU zeolite is dealuminated.

6. The process of claim 2 wherein the 10-member ring MFI zeolite is modified by steaming.

7. The process of claim 1 wherein the amination catalyst has acid sites in the range of 0.40 to 1.15 mmol/g.

8. The process of claim 1 wherein the amination catalyst has a mole ratio of Si to Al in a range of 5-140.

9. The process of claim 1:
wherein the amination reaction takes place at a temperature in a range of 280° C. to 350° C., a pressure in a range of 1700-2500 psig, and a molar ratio of NH₃ to feed) of 10 to 100; or
wherein the cyclization reaction takes place at a temperature in a range of 280° C. to 350° C., and a pressure in a range of 1700-2500 psig.

10. The process of claim 1 wherein the cyclization catalyst comprises a 10-member ring zeolite.

11. The process of claim 10 wherein the 10-membered ring zeolite comprises a 10-member ring MFI zeolite.

12. The process of claim 10 wherein the 10-member ring zeolite is modified with gallium, or by silylation, or by steaming, or combinations thereof.

13. The process of claim 1 wherein the alkanolamine comprises diethanolamine, or monoethanolamine, or triethanolamine, or monoisopropanolamine, or aminoethylethanolamine, combinations thereof.

14. The process of claim 1 wherein the diol comprises ethylene glycol or monopropylene glycol or both.

15. The process of claim 1 wherein the feed stream further comprises a C3 analog of the alkanolamine or the diol or both, wherein the aminated feed stream further comprises an aminated C3 analog, and wherein the reaction mixture further comprises an alkylated piperazine.

16. The process of claim 15 wherein the C3 analog comprises monopropylene glycol or monoisopropanol amine or both.

17. The process of claim 1 wherein the reaction mixture further comprises a piperazine derivative and further comprising:
separating the reaction mixture into a piperazine stream comprising the piperazine and a byproduct stream comprising the piperazine derivative; and
recycling the byproduct stream to the cyclization reaction zone.

18. The process of claim 17 wherein the piperazine derivative comprises at least one of a heavy alkylated piperazine, triethylenediamine, aminoethylpiperazine, and hydroxyethylpiperazine.

19. The process of claim 1 further comprising:
co-feeding anhydrous NH₃ to the cyclization reaction zone; and
wherein the cyclization reaction takes place at a temperature in a range of 280° C. to 350° C., a pressure in a range of 1700-2500 psig, and a molar ratio of NH₃ to feed of 10 to 100.

20. A process for the production of piperazine comprising:
aminating a feed stream comprising an alkanolamine, or a diol, or both, and optionally a C3 analog of the alkanolamine or the diol in an amination reaction zone comprising an amination reactor in the presence of anhydrous ammonia and an amination catalyst to provide an aminated feed stream comprising an aminated alkanolamine, or an aminated diol, or both, and optionally an aminated C3 analog of the alkanolamine or the diol or both, wherein the amination catalyst comprises a 10-member ring MFI zeolite, a 12-member ring FAU zeolite, or amorphous silica-alumina;
cyclizing the aminated feed stream in a cyclization reaction zone comprising a cyclization reactor in the presence of a cyclization catalyst comprising a 10-member ring zeolite to produce a reaction mixture comprising piperazine, and optionally an alkylated piperazine, and optionally a piperazine derivative; and
separating the reaction mixture into a piperazine stream comprising the piperazine and optionally the alkylated piperazine, and a byproduct stream comprising the piperazine derivative.
